(19) United States
(12) Patent Application Publication
(10) Pub. No.: US 2015/0209756 A1
(43) Pub. Date: Jul. 30, 2015

(54) MAGNETIC CORE-SHELL PARTICLES WITH HIGH SEPARATION EFFICIENCY

(71) Applicants: Stipan Katusic, Bad Soden (DE); Peter Kress, Karlstein (DE); Juergen Meyer, Stockstadt (DE); Aymee Lisette Michel De Arevalo, Bruchkoebel (DE); Harald Herzog, Karlstein (DE)

(72) Inventors: Stipan Katusic, Bad Soden (DE); Peter Kress, Karlstein (DE); Juergen Meyer, Stockstadt (DE); Aymee Lisette Michel De Arevalo, Bruchkoebel (DE); Harald Herzog, Karlstein (DE)

(73) Assignee: Evonik Industries AG, Essen (DE)

(21) Appl. No.: 14/413,608

(22) PCT Filed: Jun. 18, 2013
(86) PCT No.: PCT/EP2013/062558

§ 371 Date: Jan. 8, 2015

(30) Foreign Application Priority Data
Jul. 9, 2012 (DE) 10 2012 211 947.7

(51) Int. Cl. B01J 20/22; B05D 1/02; B01J 20/32; C12N 11/02; B01J 20/28; B01J 20/06

(52) U.S. Cl. CPC B01J 20/22; B01J 20/28009; B01J 20/28016; B01J 20/06; B01J 20/3293; C12N 11/02; B05D 1/02

(57) ABSTRACT

Functionalized magnetic core-shell particles which are present predominantly in the form of isolated, essentially spherical individual particles, the core of which consists essentially of one or more magnetic iron oxides, the shell of which consists essentially of impervious, amorphous silicon dioxide, and the functionalization of which consists of amino or epoxy group units on the surface of the particles, and which additionally
have a mean particle diameter d₅₀ such that 2<d₅₀<10 μm,
the particles have a content of iron oxide of 83 to 92% by weight, of silicon dioxide of 5 to 15% by weight, and of carbon of 0.5 to 3% by weight,
the amino or epoxy group is part of the structural unit —OSi-alkyl-X where X is NH₂ or epoxy and alkyl is C₂-C₈, and
the concentration of the amino groups or of the epoxy groups is at least 30 μmol/g of particles.

The particles are used for immobilization of enzymes.

The invention relates to magnetic core-shell particles and to surface-modified magnetic core-shell particles with high separation efficiency to the production thereof and to the use thereof.

WO03/042315 discloses adhesive bonds comprising inductively heatable core-shell particles with a core of inductively excitable materials and a shell of silicon dioxide. These can be produced via sol-gel processes or from the reaction of nanoscale iron oxide with sodium waterglass. The average primary particle size is less than 1 μm, more preferably 0.002 to 0.1 μm.

WO2010/063557 discloses iron-silicon oxide particles which can be used for inductive heating of materials in a magnetic or electromagnetic alternating field. The particles have a core-shell structure, with iron oxides as the core and an amorphous shell of silicon dioxide, and have a mean particle diameter of 5 to 100 nm.

DE-A-102008001433 discloses a hydrophobized magnetic mixed silicon-iron oxide powder having a BET surface area of 20 to 75 m²/g and a particle size of 2 to 200 nm. The reactant used is a mixed silicon-iron oxide powder in the form of aggregated primary particles consisting of spatially separate regions of silicon dioxide and iron oxide.

WO01/88540 discloses silicon dioxide-coated magnetic nanoparticles, the mean diameter of which is less than 1 μm. These can be surface-modified by reaction with a silanizing agent and can serve for immobilization of biomolecules.

The particles mentioned in the prior art have the disadvantage that they are often too small when used in processes in which a separation of these particles from a reaction medium is required as a final reaction step, and the concentration of the functional groups bound to the surface by modification is too small to immobilize biomolecules, for example enzymes, in a desired amount.

The technical object of the present invention therefore consisted in providing magnetic particles which have greater particle dimensions compared to the prior art and a high concentration of bound functional groups.

The invention provides functionalized magnetic, for example ferrimagnetic, ferromagnetic or superparamagnetic, core-shell particles
a) which are present predominantly in the form of isolated, essentially spherical individual particles,
b) the core of which consists essentially of one or more magnetic iron oxides,
c) the shell of which consists essentially of impervious amorphous silicon dioxide,
d) the functionalization of which consists of amino or epoxy group units on the surface of the particles, wherein
e) for the mean particle diameter d₅₀, 2<d₅₀<10 μm,
f) the particles have a content of iron oxides of 83 to 92% by weight, of silicon dioxide of 5 to 15% by weight and of carbon of 0.5 to 3% by weight, the sum of these constituents being at least 98% by weight, based on the functionalized magnetic core-shell particles,
g) the amino or epoxy group is part of the structural unit —OSi-alkyl-X where X is NH₂ or epoxy and alkyl is C₂-C₈, where the alkyl radical may be linear or branched, and optionally have one or more oxygen and/or nitrogen atoms, preference being given to —OSi-(CH₂)₃NH₂ or

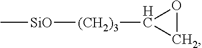

and
h) the concentration of the amino groups or of the epoxy groups is at least 30 μmol/g of the inventive particles.

The core-shell structure of the inventive particles can be detected, for example, by means of TEM (Transmission Electron Microscopy). TEM also shows that the inventive particles are predominantly in the form of isolated individual particles. “Predominantly” is understood to mean that, in the case of counting of about 1000 to 2000 particles in a TEM image, at least 70%, preferably at least 80%, more preferably at least 90%, most preferably at least 98%, are in the form of isolated individual particles, and the rest are each in the form of aggregated particles, where at least two individual particles are firmly fused to one another. The inventive particles show an essentially spherical appearance in the TEM. “Essentially” is intended to mean that ellipsoidal or bulb-shaped particles may also be present, but no acicular particles, for example.

The d₅₀ can be determined from the image counting of TEM images. The d₅₀ is understood to mean the median of the weight distribution. Preference is given to a d₅₀ of 3 to 7 μm.

The concentration of the amino groups or of the epoxy groups of the inventive core-shell particles is at least 30 μmol/g of particles. In the case of modification of the particles with amino groups, the concentration of the amino group is preferably 100 to 200 μmol/g of particles, and the concentration of the epoxide group preferably 30 to 80 μmol/g of particles.

The BET surface area of the particles is preferably 3 to 10 m²/g.

The core of the inventive core-shell particles, in a particular embodiment, consists to an extent of 90 to 98% by weight of magnetite and to an extent of 2 to 10% by weight of at least one further ferri-, ferro- or superparamagnetic iron oxide, such as w\ddot{s}tite and/or maghemite. In addition, it is also possible for traces of amorphous iron oxide and of haematite β-Fe₂O₃ and ε-Fe₂O₃ to be present. The composition of the crystalline core constituents can be determined by x-ray diffractometry using Co—K_{α} radiation within an angle range 2Θ of 10-100°. The reflections of magnetite and of maghemite overlap very significantly. Maghemite is detectable significantly on the basis of the (110) and (211) reflections in the acute angle range. The quantitative phase analysis is executed with the aid of the Rietveld method, error approx. 10% in relative terms.

The shell of the inventive particles consists essentially of impervious, amorphous silicon dioxide. “Essentially” is intended to mean that the shell may comprise proportions of carbon. “Amorphous” is understood to mean a material for which no diffraction signals can be detected by the customary methods of x-ray diffractometry. The outer shell is an impervious shell. “Impervious” is understood to mean that, on contact of the particles with hydrochloric acid under particular reaction conditions, less than 100 ppm of iron is detectable. This involves contacting 0.33 g of the particles with 20 ml of 1 N hydrochloric acid solution at room temperature for 15 minutes. A portion of the solution is subsequently analysed for iron by means of suitable analysis techniques, for example ICP (inductively coupled plasma spectroscopy). The thickness of the shell is preferably 2 to 20 nm, more preferably 5 to 15 nm.

In addition, the inventive particles may also comprise small proportions of impurities which originate from the feedstocks and/or are process-related. In general, the proportion of impurities is not more than 2% by weight, preferably less than 1.0% by weight and more preferably less than 0.5% by weight.

The inventive magnetic core-shell particles preferably have a specific maximum magnetization Mₛ of at least 50 Am², more preferably of 55 to 80 Am² and most preferably of 60 to 70 Am² per kg of the magnetic core-shell particles. Mₛ was determined by means of an alternating gradient magnetometer (AGM) of the Micromag 2900 type from Princeton.

The invention further provides a process for producing the functionalized magnetic core-shell particles. It comprises the production of magnetic core-shell particles having hydroxyl groups on the surface thereof. These hydroxyl groups react with silane compounds bearing amino or epoxy groups to give the inventive functionalized magnetic core-shell particles. In the process,
a) in a first reaction zone, an aerosol which results from the spraying of a solution comprising at least one oxidizable iron(II) compound and a carrier gas is supplied to a flame which is formed from the reaction of a combustion gas with, generally an excess of, an oxygen-containing gas,
b) the reaction gas mixture from the first reaction zone is reacted in a second reaction zone with at least one hydrolysable silicon compound selected from the group consisting of RₐSiCl₄₋ₐ where a=0, 1, 2 or 3, or Si(OR)₄ where each R═H, CH₃, C₂H₅ and C₃H₈, in each case independently, preferably SiCl₄, Si(OC₂H₅)₄ and/or Si(OCH₃)₄, each in vaporous form or in the form of an aerosol,
c) where the amount of oxidizable iron(II) compound and oxidizable and/or hydrolysable silicon compound is selected such that the proportion of oxidizable iron(II) compound is at least 80% by weight of iron oxide, calculated as Fe₃O₄, and that of oxidizable and/or hydrolysable silicon compound not more than 20% by weight, calculated as SiO₂, based on the sum of Fe₃O₄ and SiO₂,
d) where the mean residence time of the reaction mixture in the first reaction zone is 3 to 20 s, preferably 5 to 10 s, and that in the second reaction zone is 300 ms to 10 s, preferably 500 ms to 1 s,
e) subsequently the reaction mixture, optionally cooled, preferably by feeding in water, and subsequently magnetic core-shell particles are removed in solid form from gaseous or vaporous substances and
f) the magnetic core-shell particles are treated with one or more silanes of the general formula X-alkyl-Si—Y₃ to form the functionalized magnetic core-shell particles, where

X═NH₂ or epoxy; alkyl=C₂-C₈, linear or branched, optionally having one or more oxygen or nitrogen atoms; Y═Cl or OR where R═CH₃, C₂H₅, and the proportion of silane is 2 to 10% by weight, based on the sum total of Fe₃O₄ and SiO₂.

It has been found that it is important for the process according to the invention to select the temperatures correctly in the first two reaction zones. Thus, a relatively short residence time in the first reaction stage leads to products with relatively low magnetization and relatively small particle dimensions, which are unwanted in this case. The residence time in the second reaction zone is preferably much shorter than in the first. Particular preference is given to a mean residence time in the first reaction zone of 5 to 10 s and a mean residence time in the second reaction zone of 500 ms to 1 s.

The oxidizable iron(II) compound is introduced as an aerosol. The aerosol is formed from a solution comprising the oxidizable iron(II) compound by means of a carrier gas and a two- or multiphase nozzle. The aerosol preferably has a mean droplet size of not more than 150 μm. Particular preference is given to values of 20 to 100 μm. The oxidizable iron(II) compound is introduced as an aerosol. The aerosol is formed from a solution by means of a carrier gas and a one- or two-phase nozzle. The oxidizable iron(II) compound used is preferably at least one iron(II) carboxylate and/or iron(II) alkoxide. Particular preference is given to using iron(II) salts of saturated C₄-C₁₂ alkylcarboxylic acids. Very particular preference is given to iron(II) 2-ethylhexanoate. The oxidizable iron(II) compound is preferably dissolved in an organic solvent or an organic solvent mixture. Suitable solvents or constituents of the solvent are particularly C₄-C₁₂ alkylcarboxylic acids. Very particular preference is given to 2-ethylhexanoic acid. Especially suitable is a solution in which an iron(II) salt of a saturated C₄-C₁₂ alkylcarboxylic acid is in a solvent containing the corresponding saturated C₄-C₁₂ alkylcarboxylic acid, for example iron(II) 2-ethylhexanoate in 2-ethylhexanoic acid.

The content of oxidizable iron(II) compound is preferably 20 to 60% by weight based on the solution.

In a particular embodiment of the process, a solution comprising iron(II) 2-ethylhexanoate and 2-ethylhexanoic acid is used in the first reaction zone, and Si(OC₂H₅)₄ or [—O—Si(CH₃)₂]₄ and, as the silane of the general formula X-alkyl-Si—Y₃, H₂N(CH₂)₃Si(OC₂H₅)₃, H₂N(CH₂)₂NH(CH₂)₃Si(OC₂H₅)₃ or

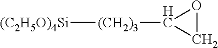

are used in the second reaction zone.

The treatment with the silanes of the general formula X-alkyl-Si—Y₃ is preferably effected by spraying them onto the as yet unfunctionalized magnetic core-shell particles, which is followed by a treatment at temperatures of 120 to 200° C., preferably under protective gas atmosphere, over a period of 1 to 5 hours.

The combustion gases used may preferably be hydrogen, methane, ethane and/or propane. Particular preference is given to hydrogen. The oxygen-containing gas used is principally air or oxygen-enriched air.

For the stability of the flame, it may be advantageous to divide the amount of air into a primary air stream and a secondary air stream. The primary air stream is supplied axially to the burner. The aerosol is sprayed into it. The secondary air stream is a stream which is preferably introduced tangentially and can contribute to an increase in the combustion rate.

The high amino or epoxy loading concentration and the high separation efficiency make it possible to use the inventive functionalized magnetic core-shell particles for immobilization of enzymes, for example from biomass.

### EXAMPLES

### Analysis

The iron oxide content is determined by digestion with NaOH, dissolution in dilute H₂SO₄ and subsequent iodometric titration. The Si content is determined by means of ICP-OES and then calculated as the oxide.

The d₅₀ is defined as the median of the numerical distribution. It is determined by image analysis by means of a Hitachi H 7500 TEM instrument and an SIS MegaView II CCD camera. The image magnification for evaluation is 30 000:1 with a pixel density of 3.2 nm. The number of particles evaluated is greater than 1000. The preparation is effected to ASTM3849-89. The lower threshold limit in relation to detection is 50 pixels.

The BET surface area is determined to DIN 66131.

The quantitative determination of the core fractions is effected by x-ray diffractometry

(reflection, θ/θ diffractometer, Cu—Kα, U=40 kV, I=35 mA; scintillation counter, downstream graphite monochromator; angle range (2Θ)/step width/measurement time: 10-100°/0.04°/6 s (4 h)). With the aid of the Rietveld method, a quantitative phase analysis is performed (error approx. 10% in relative terms). The quantitative phase analysis is effected using set 60 of the ICDD database PDF4+ (2010). The quantitative phase analysis and the crystal size determination are effected with the Rietveld programme SiroQuant®, Version 3.0 (2005).

The thickness of the shell is determined by means of high-resolution transmission electron microscopy (HR-TEM).

NH₂ loading: the solid is suspended in acetic acid and then titrated with a standard perchloric acid solution with potentiometric end point detection. The analysis result is based on the starting sample weight, and the molar amount of titrated base is reported as the amino group concentration (—NH₂) as a molar figure. The titration covers the amino group concentration accessible to the titrant (HClO₄) in suspension.

Epoxide loading: the epoxide moieties are determined by means of titration with perchloric acid in anhydrous medium. For this purpose, two perchloric acid titrations are conducted, one titration with addition of tetraethylammonium bromide, covering the epoxide groups and any basic substances present in the sample as a cumulative parameter. In a second perchloric acid titration without addition of tetraethylammonium bromide, exclusively and only the basic substances potentially present in the sample are covered. If the difference between the results of the two titrations is then found, the actual content of epoxide groups in the respective sample is obtained.

The samples are all aqueous suspensions. The solid was separated from the water phase by centrifugation, the supernatant water was decantered, and then all samples were washed twice with aqueous acetic acid (glacial acetic acid) before the titration. The solids were separated from the glacial acetic acid once again by centrifugation. After the last wash step, the solid is suspended in 50 ml of glacial acetic acid and titrated against 0.1 N perchloric acid.

Separation efficiency: dispersions with 2 g of the inventive particles per kilogram of dispersion are produced by ultrasound dispersion (IKA-Labortechnik, Ultraturrax model T 25, 8000 rpm, 15 min).

The separation cell used was a cell having an internal diameter of 30 mm and a length of 85 mm. The magnetic field can be induced by an electromagnet or permanent magnet.

To determine the feed and filtrate concentrations, the turbidity is determined.

Suitable instruments for this purpose are, for example, Hach Portable Turbidimeter Model 2100P or Optek 112/AF10 concentration measurement system. The flow rate is determined from the increase in mass of the collecting vessel. The slope of the mass signal over time is the mass flow rate, which in turn, based on the filter inflow area and density of the fluid, gives the flow rate.

### Example 1

An aerosol which is obtained by spraying 2.6 kg/h of a solution consisting of 46% by weight of iron(II) 2-ethylhexanoate, 14% by weight of 2-ethylhexanoic acid and 40% by weight of n-octane with 4.0 kg/h of N₂ by means of a two-phase nozzle, and 4 m³ (STP)/h of hydrogen and 20 m³ (STP)/h of air, of which 15 m³ (STP)/h is primary air and 5 m³ (STP)/h is secondary air, is reacted in a first zone. The mean residence time of the reaction mixture in the first zone is approx. 6.5 s. A mixture of 0.19 kg/h of vaporous Si(OC₂H₅)₄ and 2.2 kg/h of water vapour is introduced into the stream of the reaction mixture from the first zone. The mean residence of the reaction mixture in the second zone is 750 ms.

Subsequently, the reaction mixture is cooled and the solid obtained is separated from the gaseous substances on a filter.

100 parts by weight of the solid are initially charged in a mixer and sprayed with 7 parts by weight of AMEO with vigorous mixing. The end of spraying is followed by heat treatment at 130° C. over a period of 2 hours.

Examples 2 and 3 are executed analogously to Example 1. The amounts of feedstock and the reaction conditions are shown in Table 1. The physicochemical values for the solids obtained are shown in Table 2.

The separation efficiency of the inventive particles from Examples 1 to 3 is>99%.

TABLE 1
Feedstocks and reaction conditions

| Example | | 1 | 2 | 3 |
|---|---|---|---|---|
| Iron(II) 2-ethylhexanoate | kg/h | 2.6 | 2.6 | 2.6 |
| solution | | | | |
| Atomizer gas^{a)} | m³ (STP)/h | 4.0 | 4.5 | 2.0 |
| Hydrogen | m³ (STP)/h | 4 | 4 | 4 |
| Primary air | m³ (STP)/h | 15 | 15 | 14 |
| Secondary air | m³ (STP)/h | 5 | 8.5 | 6 |
| Si(OC₂H₅)₄ | kg/h | 0.19 | 0.19 | 0.18 |
| Mean residence time | | | | |
| Reaction zone 1 | s | 6.5 | 5.5 | 7.4 |
| Reaction zone 2 | ms | 750 | 630 | 950 |
| Aminosilane/epoxysilane^{b)} | | AMEO | DAMO | GLYMO |
| Content | g/100 g^{c)} | 7 | 7 | 7 |
| Reaction temperature | ° C. | 130 | 130 | 130 |
| Reaction time | h | 2 | 3 | 2 |

^{a)}Nitrogen;
^{b)}AMEO = 3-aminopropyltriethoxysilane; DAMO = aminoethyl-3-amino-propyltrimethoxysilane; GLYMO = 3-glycidoxypropyltrimethoxysilane;
^{c)}Grams of aminosilane or epoxysilane per 100 grams of unfunctionalized core-shell particles.

TABLE 2
Physicochemical data

| Example | | 1 | 2 | 3 |
|---|---|---|---|---|
| Iron oxide | % by wt. | 84.1 | 83.3 | 86.1 |
| SiO₂ | % by wt. | 14.8 | 15.0 | 13.2 |
| Carbon | % by wt. | 1.1 | 1.7 | 0.7 |
| Proportions in core of | | | | |
| magnetite | % by wt. | 96 | 91 | 90 |
| wüstite | % by wt. | 0 | 3 | 2 |
| maghemite | % by wt. | 1 | 0 | 2 |
| haematite | % by wt. | 3 | 4 | 6 |
| BET surface area | m²/g | 6 | 5 | 7 |
| Median particle diameter d₅₀ | μm | 3.02 | 3.84 | 5.60 |
| Shell thickness | nm | 5 | 6 | 8 |
| NH₂ loading | μmol/g | 119 | 156 | — |
| Epoxide loading | μmol/g | — | — | 41 |
| Magnetization Mₛ | Am²/kg | 64.2 | 66.8 | 66.3 |
| Separation efficiency | % | >99.2 | >99.4 | >99.4 |

1. Magnetic core-shell particles
which are present in the form of isolated, spherical individual particles,
wherein the particles comprise a core and a shell, the core of which consists essentially of a magnetic iron oxide,
the shell of which consists essentially of impervious amorphous silicon dioxide,
and the particles having a functionalization which consists of amino or epoxy group units on a surface of the particles,
wherein
a mean particle diameter d₅₀, is 2<d₅₀<10 μm,
the particles have a content of iron oxides of 83 to 92% by weight, of silicon dioxide of 5 to 15% by weight and of carbon of 0.5 to 3% by weight, the sum of these constituents being at least 98% by weight, based on the functionalized magnetic core-shell particles,
the amino or epoxy group is part of a structural unit —OSi-alkyl-X where X is NH₂ or epoxy and alkyl is C₂-C₈, and
a concentration of the amino groups or of the epoxy groups is at least 30 μmol/g of functionalized magnetic core-shell particles.

2. The magnetic core-shell particles according to claim 1, wherein
—OSi-alkyl-X is —OSi-(CH₂)₃NH₂ or

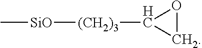

3. The magnetic core-shell particles according to claim 1, wherein
the concentration of the NH₂ group is from 100 to 200 μmol/g and the concentration of the epoxy group is from 30 to 80 μmol/g of functionalized magnetic core-shell particles.

4. The magnetic core-shell particles according to claim 1, wherein
the core consists to an extent of 90 to 98% by weight of magnetite and to an extent of 2 to 10% by weight of a further ferri-, ferro- or superparamagnetic iron oxide.

5. The magnetic core-shell particles according to claim 1, wherein a specific maximum magnetization Mₛ thereof is at least 50 Am² per kg of the functionalized magnetic core-shell particles.

6. A process for producing the magnetic core-shell particles according to claim 1, comprising
a) supplying, in a first reaction zone, an aerosol which is obtained from the spraying of a solution comprising an oxidizable iron(II) compound and a carrier gas, to a flame which is formed from the reaction of a combustion gas with, an excess of, an oxygen-containing gas to obtain a reaction gas mixture;
b) reacting the reaction gas mixture from the first reaction zone in a second reaction zone with at least one hydrolysable silicon compound selected from the group consisting of RₐSiCl₄₋ₐ where a=0, 1, 2 or 3, and Si(OR)₄ where each R═H, CH₃, C₂H₅ or C₃H₈, in each case independently, each in vaporous form or in the form of an aerosol,
wherein an amount of oxidizable iron(II) compound and oxidizable and/or hydrolysable silicon compound is selected such that a proportion of oxidizable iron(II) compound is at least 80% by weight of iron oxide, calculated as Fe₃O₄, and that of oxidizable and/or hydrolysable silicon compound is from 3 to 20% by weight, calculated as SiO₂, based on the sum of Fe₃O₄ and SiO₂,
a mean residence time of the reaction mixture in the first reaction zone is from 3 to 20 s, and a mean residence time of the reaction mixture in the second reaction zone is from 300 ms to 10 s;
c) subsequently removing the reaction mixture and subsequently the magnetic core-shell particles in solid form from gaseous or vaporous substances; and
d) treating the magnetic core-shell particles with a silane of formula X-alkyl-Si—Y₃ to form the functionalized magnetic core-shell particles, where
X═NH₂ or epoxy; alkyl=C₂-C₈, linear or branched, optionally having an oxygen or nitrogen atom; and Y═Cl or OR where R═CH₃, C₂H₅,
and a proportion of silane is from 2 to 10% by weight, based on the sum total of Fe₃O₄ and SiO₂.

7. The process according to claim 6, wherein a solution comprising iron(II) 2-ethylhexanoate and 2-ethylhexanoic acid is employed in the first reaction zone,
and Si(OC₂H₅)₄ or [—O-Si(CH₃)₂]₄ and, as the silane of formula X-alkyl-Si—Y₃, H₂N(CH₂)₃Si(OC₂H₅)₃, H₂N(CH₂)₂NH(CH₂)₃Si(OC₂H₅)₃ or 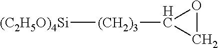
are employed in the second reaction zone.

8. The process according to claim 6, wherein the silane of formula X-alkyl-Si—Y₃ is sprayed onto the magnetic core-shell particles and then treated at a temperature of from 120 to 200° C., over a period of 1 to 5 hours.

9. A process, comprising immobilizing enzymes with the magnetic core-shell particles according to claim 1.
